(19) United States
(12) Patent Application Publication
(10) Pub. No.: US 2014/0148632 A1
(43) Pub. Date: May 29, 2014

(54) RESID CATALYTIC CRACKER AND CATALYST FOR INCREASED PROPYLENE YIELD

(71) Applicant: UOP LLC, Des Plaines, IL (US)

(72) Inventors: Robert L. Mehlberg, Wheaton, IL (US); Erick D. Gamas-Castellanos, Houston, TX (US); Chad R. Huovie, Park Ridge, IL (US); Christopher P. Nicholas, Evanston, IL (US)

(73) Assignee: UOP LLC, Des Plaines, IL (US)

(21) Appl. No.: 14/169,236

(22) Filed: Jan. 31, 2014

Related U.S. Application Data
(63) Continuation-in-part of application No. 13/300,091, filed on Nov. 18, 2011.

(51) Int. Cl. C07C 4/06; B01J 29/46; B01J 29/40

(52) U.S. Cl. CPC C07C 4/06; B01J 29/40; B01J 29/405; B01J 29/46
USPC 585/653; 502/77; 502/73; 502/69; 502/74

(57) ABSTRACT

A process and catalyst for improving the yield of propylene from residual oil feedstock includes obtaining residual oil feedstock from a vacuum distillation tower. The residual oil feedstock has contaminant metals such as sodium or vanadium. The residual oil feedstock is contacted with a cracking catalyst in a catalytic cracking zone to make products. A ZSM-5 zeolite, a binder, a filler and a metal trap are components of the cracking catalyst. The metal trap has a trapping agent in an outer shell of the catalyst, a trapping agent in the ZSM-5 binder or combinations thereof. After reacting, the cracking catalyst is separated from the products in a separator zone, then regenerated by combusting coke deposited on a surface of the cracking catalyst in an oxygen-containing environment. The cracking catalyst is returned to the catalytic cracking zone. The catalyst with the metal trap is also disclosed.

### CROSS-REFERENCE TO RELATED APPLICATION

This application is a Continuation-In-Part of copending application Ser. No. 13/300,091 filed Nov. 18, 2011, the contents of which are hereby incorporated by reference in its entirety.

### FIELD OF THE INVENTION

This invention relates to increasing yields of propylene in refinery processes. More specifically, it relates to a process and a catalyst for increasing the yield of propylene from feedstocks high in metal content.

### BACKGROUND OF THE INVENTION

In typical refinery operations, residual oil (“resid”) is produced in a vacuum distillation column. As demand for petroleum fuels and petrochemicals increases, it becomes more important to process feedstocks, such as resids, that are typically low in value. In one process for converting resids to more valuable products, the resid is fed to a resid catalytic cracking zone (“RFCC”) to produce naphtha and distillate fuels which are higher in value. Cracking of the high boiling resid to lighter hydrocarbon streams, such as naphtha or diesel oils, thus generates additional profit due to the relative economic value of the hydrocarbon streams.

One reason resids are difficult to process is because of its high contaminant metals content. Contaminant metals, such as nickel, vanadium and sodium, are known to deactivate the processing catalysts. The metal deactivates the catalyst in one of many ways. In some processes, the contaminant metal is attracted to active reaction sites on the catalyst and physically blocks them. In other cases, the contaminant metal blocks access to pores or cavities in the catalyst support in which the reactions occur. Contaminant metals also reduce the hydrothermal stability of some refining catalysts. Upon exposure to the high temperatures, such as those observed in commercial FCC regenerators, contaminant metals can cause an increased tendency for catalysts to sinter and become amorphous in nature, resulting in a loss of catalyst activity. This is a particular problem in catalytic cracking processes where regenerator temperatures reach as high as 705° C. (1300° F.).

Where a number of catalytic reactions are competing for different sites on the catalyst surface, contaminant metals can alter the catalyst selectivity as well as the activity. Where, for example, a catalyst has both acidic and metallic reaction sites, the contaminant metal could be more attracted to one of these sites over the other. The product distribution will change where the relative activity of one of the two sites changes compared to the other. Thus, both activity and selectivity of the catalyst is altered in the presence of contaminant metals.

Another problem with feedstocks having contaminant metals is that the metals are not removed during catalyst regeneration. Regeneration removes carbon, or “coke”, deposits by combustion. However, this technique does little to remove some contaminant metals.

Methods are known to mitigate the effects of contaminant metals. One basic method is removal of at least some of the contaminant metals from the catalyst. Other methods, known as metals passivation, leave the metals in place, but reduce the negative effects that they have on the activity and selectivity of the catalyst.

### SUMMARY OF THE INVENTION

These and other needs are at least partially addressed by the invention described herein. A process for improving the yield of propylene from residual oil feedstock includes obtaining residual oil feedstock from a vacuum distillation tower. The residual oil feedstock has contaminant metals such as sodium or vanadium. The residual oil feedstock is contacted with a cracking catalyst in a catalytic cracking zone to make products. A ZSM-5 zeolite, a core, an outer shell comprising a layer surrounding the core, and a metal trap are components of the cracking catalyst. The metal trap comprises a first trapping agent in the core and a second trapping agent in the outer shell on the surface of the catalyst. After reacting, the cracking catalyst is separated from the products in a separator zone, then regenerated by combusting coke deposited on a surface of the cracking catalyst in an oxygen-containing environment. The cracking catalyst is returned to the catalytic cracking zone.

In another embodiment, the residual oil feedstock is contacted with a first portion of the cracking catalyst in a catalytic cracking zone to make a first slate of products, the cracking catalyst including a ZSM-5 zeolite, a core, an outer shell surrounding the core on the surface of the catalyst and a metal trap, wherein the metal trap comprises a first trapping agent in the core, a second trapping agent in an outer shell. The cracking catalyst is separated from the first slate of products, including a light naphtha, in a first separator zone. After separation, the light naphtha is contacted with a second portion of the cracking catalyst to make a second slate of products in an olefin-producing zone. Separation of the second portion of the cracking catalyst takes place in a second separator zone. After separation, the cracking catalyst is regenerated by combusting coke deposited on a surface of the first portion of the cracking catalyst in an oxygen-containing environment and returning the first portion of the cracking catalyst to the catalytic cracking zone and the second portion of the cracking catalyst.

In an embodiment, a catalyst composition comprises a ZSM-5 zeolite, a core, an outer shell surrounding the core on the surface of the catalyst and a metal trap, wherein the metal trap comprises a first trapping agent in the core, a second trapping agent in an outer shell.

Reducing the effect of contaminant metals has a beneficial effect on yields of light olefins, such as ethylene and propylene. Utilizing a ZSM-5 cracking catalyst with a metal trap prevents the contaminant metals from reducing the activity and altering the selectivity of the cracking catalyst.

### BRIEF DESCRIPTION OF THE DRAWINGS

FIG. 1 is a schematic front plan view of one embodiment of the present resid catalytic cracking process;

FIG. 2 is a schematic front plan view of an alternative embodiment of the present resid catalytic cracking process.

### DETAILED DESCRIPTION OF THE INVENTION

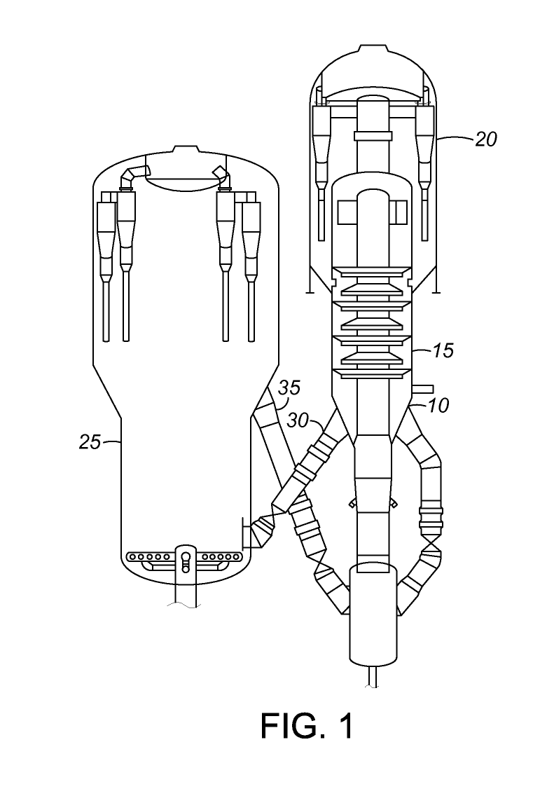

A light naphtha feedstock is obtained from a resid catalytic cracking zone in a process for increasing propylene yield. Any catalytic cracking process or apparatus may be used as the primary cracking zone, including of those adapted to process catalytic cracking feedstocks obtained from atmospheric or vacuum residual oils. An example of this process includes a resid catalytic cracking process (“RFCC”), licensed by UOP, LLC, as shown in FIG. 1.

In some exemplary embodiments, the catalytic cracking zone uses a fluidized bed process. The cracking catalyst is combined with a lift gas and a catalytic cracker feedstock at the entrance to a first riser reactor 10. The catalytic cracker feedstock is a resid having vanadium, sodium or combinations thereof. It is also contemplated that the feedstock contains additional metals, including, but not limited to nickel and zinc. Vanadium is present in amounts of from about 20 ppm to about 400 ppm, preferably in amounts of about 20 ppm to about 150 ppm and more preferably at least 30 ppm. Sodium is present in amounts of from about 1 ppm to about 15 ppm, preferably in amounts of about 1 ppm to about 10 ppm, and more preferably in amounts of at least 5 ppm. Other characteristics of the resid feedstock are: boiling range 340° C. (644° F.) to 566° C. (1050° F.), API in the range of 6 to 21, total sulfur content in the range of 0.3 wt % to 8.0 wt % and microcarbon residue (Conradson carbon) in the range of 4.0 wt % to 16 wt %.

As it travels the length of the riser reactor 10, the catalytic cracker feedstock reacts in the presence of the cracking catalyst to generate a first slate of products lower in molecular weight than the catalytic cracking feedstock. Typical products include light cycle oils, naphtha, and a light ends stream. For the purposes of this invention, light naphtha is considered to be C₅-C₆ hydrocarbons from the naphtha, light cycle oil is C₇-C₁₂ hydrocarbons, cycle oils include hydrocarbons heavier than C₁₂ and the light ends stream includes C₄-hydrocarbons.

In the catalytic cracking zone 15, the catalytic cracker feedstock is contacted with a cracking catalyst that promotes cracking of heavy oils to lighter, more valuable, products. Catalysts that promote cracking include, but are not limited to, large and medium pore molecular sieves. The cracking catalyst includes a ZSM-5 zeolite component. U.S. Pat. No. 3,702,886, herein incorporated by reference, describes the ZSM-5 zeolite and its preparation in greater detail. Preferably, the ZSM-5 zeolite is dispersed on a matrix that includes a binder material, such as silica or alumina, and an inert filler material, such as kaolin. These catalyst compositions have a crystalline ZSM-5 zeolite content of 10 to 25 wt-% or more and a matrix material content of 75 to 90 wt-%. Catalysts containing about 25 wt-% crystalline ZSM-5 zeolite materials are preferred. Greater crystalline zeolite content may be used in this catalyst, provided they have satisfactory attrition resistance. The cracking zone catalyst may also comprise another active material such as Beta zeolite.

The cracking catalyst may be a layered composition comprising an inner core and an outer shell or layer on the outer surface of the catalyst surrounding the core. The inner core can be formed into a variety of shapes such as pellets, extrudates, spheres or irregularly shaped particles. Preparation of the inner core can be done by means known in the art such as oil dropping, pressure molding, metal forming, pelletizing, granulation, extrusion, rolling methods and marumerizing. A spherical inner core is preferred. The outer layer is applied to the core, so the finished layered catalyst will have a shape that approximates the shape of the core. The outer layer and the core should have different compositions.

In addition to the ZSM-5 zeolite, binder and filler, the cracking catalyst also includes a component that acts as a metal trap for the contaminant metals. As described herein, a “metal trap” is a component of the cracking catalyst that stops the contaminant metal from interfering with the catalyst's activity or selectivity. The metal trap acts as a decoy to bind the contaminant metal to the metal trap preferentially over the ZSM-5 zeolite. This limits reduction in activity and selectivity of the ZSM-5 zeolite due to deactivation of the zeolite by the contaminant metals. The metal trap includes one or more trapping agents as part of the metal trap component. Each of three embodiments of the metal trap is described below. It is contemplated that they be used individually or in any combination with one another. These catalysts are fluidizable such that they may be utilized in a FCC process. Characteristics of fluidizable catalysts such as density, particle size, particle size distribution and the like are well known in the art such as those given in U.S. Pat. No. 5,012,026.

An embodiment of the metal trap includes the addition of a first trapping agent to the inner core of the ZSM-5 catalyst. When present, the first trapping agent is present in amounts of from about 0.5 wt % to about 25 wt %. Possible first trapping agents include cerium, cerium compounds and active alumina. The cerium may be preferably present in the form of ceria, CeO₂ in the finished catalyst. Amounts of cerium may comprise from about 0.5 wt % to about 50 wt % in the ZSM-5 catalyst, and preferably from about 0.5 wt % to about 15 wt %. A preferred binder for cerium addition is alumina. In an embodiment, the first trapping agent may be present in the core of the ZSM-5 catalyst particle. That is, the concentration of the first trapping agent may not be constant throughout the catalyst particle. Preferably the content of the first trapping agent may be higher in the core of the particle than it is in the shell of the catalyst particle. The first trapping agent may be present only in the core of the ZSM-5 catalyst particle and not present in the shell of the ZSM-5 catalyst particle. The first trapping agent may be embedded in the binder material in the core by the process of wash coat slurry impregnation. In another embodiment, the metal trap includes a second trapping agent embedded in a shell on the surface of the cracking catalyst. The second trapping agent includes phosphorous pentoxide, calcium carbonate, ferric oxide or combinations of thereof. When present, the second trapping agent is present in amounts of from about 0.5 wt % to about 30 wt %. Preferably, the calcium carbonate is present in amounts of about 0.5 wt % to about 8.0 wt %, and preferably from about 0.5 wt % to about 3.0 wt %. Amounts of ferric oxide range from about 0.1 wt % to about 30.0 wt % and preferably from about 0.1 wt % to about 10 wt %. The second trapping agent may be present only in the outer shell of the ZSM-5 catalyst particle and not present in the core of the ZSM-5 catalyst particle.

The shell may additionally comprise alumina and may be prepared by slurry wash coat impregnation the premade cores and spray drying.

In yet another embodiment of this invention, a third trapping agent is added to the bed of the ZSM-5 catalyst. A commercially available trapping agent is Cat-Aid® V, made by InterCat of Sea Girt, N.J.

Active alumina can act as a metal trap, a binder, and/or a support for other components when applied as a shell. If alumina is used as a binder, a layer of active alumina can be added on top of the alumina as a first trapping agent in the core. Preferably, if alumina is used as a binder, ceria would be used as the first trapping agent to take advantage of the higher selectivity of ceria.

In an embodiment, the first trapping agent may be incorporated into the ZSM-5 catalyst by combining the zeolite powder, a source of the first trapping agent, a source of binder and optionally a filler material; forming an aqueous slurry of the components; and spray drying the aqueous slurry to provide the catalyst cores. The cores should be a fluidizable powder suitable for use in a FCC process. If cerium is the first trapping agent, sources of cerium may include cerium acetate, cerium oxide, ammonium cerium nitrate, cerium nitrate and cerium sulfate. If the cerium source includes cerium oxide, preferably the cerium oxide has been wet-milled prior to contact with the zeolite powder. This treatment may reduce the particle size of the ceria. Sources of filler material may include clays such as kaolin or montmorillonite. If active alumina is the first trapping agent, the aqueous slurry may be spray dried without the first trapping agent, and then the spray-dried core may be wash coated with active alumina to provide the first trapping agent on the core.

In an embodiment, the second trapping agent may be incorporated into the ZSM-5 catalyst by slurry wash coat impregnating the cores comprising the first trapping agent, perhaps formed by spray drying, with a source of the second trapping agent. The slurry wash coat solution may further comprise a source of binder. Following the slurry wash coating procedure, the material may then be spray dried to yield a fluidizable powder comprising a layered composition suitable for use in an FCC process. The ZSM-5 catalyst particle may thus comprise a first trapping agent in the core of the particle and a second trapping agent in a shell on the surface of the cracking catalyst.

In an embodiment the ZSM-5 catalyst may be withheld from the materials used to make the core and just included in materials to make the outer shell along with the second trapping agent. In this embodiment, the ZSM-5 catalyst would be present in just the outer shell but not present in the core.

Optionally, the cracking catalyst includes a second molecular sieve with a large pore size. The second molecular sieve has pores with openings of greater than 0.7 nm in effective diameter defined by greater than 10- and, typically, 12-membered rings. Pore Size Indices of large pores are above about 31. Suitable large pore zeolite components include zeolites such as X-type and Y-type zeolites, mordenite and faujasite. The second molecular sieve optionally includes a metal ion on its surface, such as a rare-earth metal. Preferably, the rare earth metal is present in amounts of from about 0.1 wt % to about 3.5 wt %. It has been found that Y zeolites with low rare earth content are preferred as the cracking catalyst component. Low rare earth content denotes less than or equal to about 1.0 wt % rare earth oxide on the zeolite portion of the catalyst. Octacat™ catalyst made by W. R. Grace & Co. is an example of a suitable low rare earth Y-zeolite catalyst.

The cracking zone is operated at any useful process conditions. Temperatures range from 510° C. (950° F.) to about 594° C. (1100° F.). Pressures vary between 69 KPa (10 psi) and 276 KPa (40 psi). The space velocity (weight bases) is from about 1 hr⁻¹ to about 50 hr⁻¹. Variations in these conditions are due to differences in feedstock, catalyst and process equipment. Residence time for the catalytic cracker feedstock in contact with the cracking catalyst in the riser is from about 0.1 to 5 seconds, preferably less than or equal to 2 seconds. The exact residence time depends upon the catalytic cracker feedstock quality, the specific catalyst and the desired product distribution. Short residence time assures that the desired products are not converted to undesirable products by further reaction. Hence, the diameter and height of the riser may be varied to obtain the desired residence time.

At the top of the catalytic cracking zone 15, the cracking catalyst is separated from the catalytic cracker feedstock and the lift gas in a first separator zone 20. In a fluidized bed system, some of the cracking catalyst falls by gravity in an area of reduced pressure. One or more cyclones are optionally used to improve separation of the cracking catalyst from the catalytic cracking products.

As the primary catalytic cracking feedstock reacts with the cracking catalyst, coke deposits on the cracking catalyst covering reaction sites and causing a reduction in catalyst activity. The catalyst activity is restored by burning the cracking catalyst in the presence of oxygen from an oxygen source in a primary regeneration zone 25. The first separator zone 20 is in fluid communication with the primary regeneration zone 25, such as using a first conduit 30 to carry the cracking catalyst from the first separation zone 20 to the primary regenerator zone 25. Air is typically used as the oxygen source. As the coke burns, heat and hot combustion gases are generated. Heat generation is regulated by controlling the amount of oxygen, fuel or both provided to the primary regeneration zone. When a substantial portion of the coke has been burned from the cracking catalyst surface, the cracking catalyst is separated from the combustion gases and exits the primary regeneration zone 25. The combustion gases are removed as flue gas. In some embodiments where large amounts of coke are deposited, the cracking catalyst is cooled in a catalyst cooler as it exits the regeneration zone 25. The regeneration zone 25 is in fluid communication with the cracking zone 15, such as via a second conduit 35 that returns the regenerated cracking catalyst to the cracking zone 15.

The product effluent from the primary cracking zone is typically processed through a product recovery section, not shown. Methane, ethane, ethylene, propane, propylene, light naphtha, cycle oil naphtha, cycle oil and gas oil are all potentially part of the first slate of products recovered from the primary cracking zone. The exact products derived from the catalytic cracking process depend on the catalytic cracking feedstock selected, the exact process conditions, the cracking catalyst selected, the downstream processes that are available and the current, relative economic value of the products.

The light naphtha from the product recovery section is provided as feedstock to an olefin-producing zone. In a broad embodiment of the process of this invention, the light naphtha from the catalytic cracker is converted to light olefins in any downstream process. For the purposes of this document, light olefins are defined as C₃-olefins, including ethylene and propylene. By mitigating the effects of the contaminant metals, changes in the activity and selectivity that normally occur when contaminant metals are present in the catalytic cracking zone are reduced. The amount and composition of light naphtha are more favorable toward light olefins production when the effects of contaminant metals are limited.

In another embodiment of this invention, the olefin-producing zone is also a fluidized bed zone. The olefin catalyst is a small particle catalyst. The light naphtha feedstock is contacted with the olefin catalyst in the olefin-producing zone. In a second riser reactor, the olefin catalyst is entrained in the hydrocarbon gases and a lift gas as they move up the riser. At the end of the second riser, a second slate of products, which is now rich in olefins such as propylene, and the entrained olefin catalyst enter a second separation zone and are separated. The olefin-rich product hydrocarbons are drawn from the top of the second separation zone while the olefin catalyst falls away by gravity. Any equipment that can effect such a separation may be used, including, but not limited to, cyclone separators as described above. Following separation of the olefin catalyst, the olefin-rich product exits the second separation zone. The second separation zone is in fluid communication with a regenerator for the olefin catalyst. One example of this fluid communication is a third conduit for transfer of the olefin catalyst from the second separator zone to the regenerator.

The light naphtha feedstock to the olefin-producing zone reacts in the presence of an olefin catalyst. Any known olefin catalyst can be used. In some embodiments of this invention, the cracking catalyst is also used as the olefin catalyst. In other embodiments, the olefin catalyst has a greater selectivity for olefin production than the cracking catalyst and the cracking catalyst has a higher selectivity for cracking than the olefin catalyst.

Any useful process conditions can be utilized in the olefin-producing zone. Temperatures range from 510° C. (950° F.) to about 594° C. (1100° F.). Pressures vary between 69 KPa (10 psi) and 276 KPa (40 psi). The space velocity (weight bases) is from about 1 hr⁻¹ to about 50 hr⁻¹. Variations in these conditions are due to differences in feedstock, catalyst and process equipment. In preferred embodiments, the olefin-producing zone is approximately 34 KPa (5 psi) lower in pressure than the pressure in the catalytic cracking zone. Residence time for the light naphtha feed in contact with the olefin catalyst in the riser is from about 0.1 to 5 seconds, preferably less than or equal to 2 seconds. The exact residence time depends upon the feedstock quality, the specific olefin catalyst and the desired product distribution. Short residence time assures that the desired products, such as light olefins, do not convert to undesirable products in subsequent reactions. Hence, the diameter and height of the riser may be varied to obtain the desired residence time.

Coke builds up on the olefin catalyst as well as the cracking catalyst, and must be burned off to restore catalyst activity. In another embodiment of the invention, the olefin catalyst is regenerated in a second regeneration zone similar to that of the first regeneration zone. Process conditions are selected from the same ranges as the first regeneration zone. The regenerator for the olefin catalyst is in fluid communication with the olefin-producing zone for transfer of the olefin catalyst back to the olefin-producing zone, as by a fourth conduit.

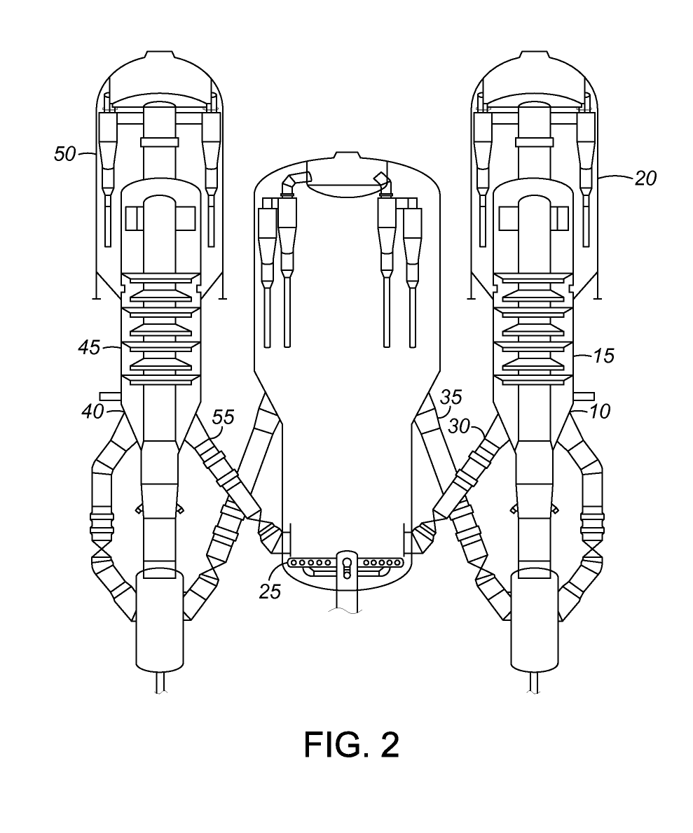

In yet another embodiment of the invention as shown in FIG. 2, the cracking zone 15 and the olefin-producing zone 45 of the second riser reactor 40 use a first portion and a second portion of the same cracking catalyst and share the primary regeneration zone 25. In this case, following regeneration, the first portion of the cracking catalyst is returned to the cracking zone 15. The second portion of catalyst from the primary regeneration zone 25 is fed to the olefin-producing zone 45. In the olefin-producing zone 45, the cracking catalyst contacts the light naphtha feedstock at olefin-producing conditions to make the second slate of light olefin products, such as propylene and ethylene. The second slate of olefin products is separated from the cracking catalyst in the second separator zone 50. The second separator zone 50 is in fluid communication with the primary regeneration zone 25 by a conduit 55 for transfer of the cracking catalyst thereto.

In the primary regeneration zone 25 the first portion of the cracking catalyst comingles with the second portion of the cracking catalyst in a common cracking catalyst pool. Regeneration of the common catalyst pool takes place in the manner described above. Any known catalytic cracking process conditions may be used. Any method may be used to separate the common catalyst pool into a first portion and a second portion. Preferably, the bottom of the primary regeneration zone 25 is shaped so as to divide the common catalyst pool by gravity. It will be understood by those of ordinary skill in the art that “the first portion” and “the second portion” of the cracking catalyst refer to amounts of catalyst only. Once comingled in the common cracking catalyst pool, individual particles are fed to the first portion and second portion of the cracking catalyst randomly

It is important in this embodiment to mitigate the effects of the contaminant metals. Where there is a common regeneration zone, metals build up on the first portion of cracking catalyst in the cracking zone. However, comingling of the first portion and the second portion in the regeneration zone allows the second portion of cracking catalyst to include catalyst particles that have contaminant metals from prior passes through the cracking zone.

It is to be understood that the features of any of the embodiments discussed above may be recombined with any other of the embodiments or features disclosed herein. While particular features and embodiments of a process and reactor system for increasing propylene yields has been shown and described, other variations of the invention will be obvious to those of ordinary skill in the art. All embodiments considered to be part of this invention are defined by the claims that follow.

### Specific Embodiments

While the following is described in conjunction with specific embodiments, it will be understood that this description is intended to illustrate and not limit the scope of the preceding description and the appended claims.

A first embodiment of the invention is a process for improving the yield of propylene from a light naphtha feedstock, comprising obtaining residual oil feedstock from an atmospheric or vacuum distillation tower, the residual oil feedstock comprising contaminant metals; contacting the residual oil feedstock with a cracking catalyst in a catalytic cracking zone to make a slate of products, the cracking catalyst comprising a ZSM-5 zeolite, a core, an outer shell comprising a layer surrounding the core on the surface of the catalyst and a metal trap, wherein the metal trap comprises a first trapping agent in the core, a second trapping agent in the outer shell; separating the cracking catalyst from the products in a separator zone; regenerating the cracking catalyst by combusting coke deposited on a surface of the cracking catalyst in an oxygen-containing environment; and returning the cracking catalyst to the catalytic cracking zone. An embodiment of the invention is one, any or all of prior embodiments in this paragraph up through the first embodiment in this paragraph further comprising a third trapping agent added to a bed of cracking catalyst. An embodiment of the invention is one, any or all of prior embodiments in this paragraph up through the first embodiment in this paragraph wherein the shell further comprises an alumina wash coat. An embodiment of the invention is one, any or all of prior embodiments in this paragraph up through the first embodiment in this paragraph wherein the second trapping agent comprises phosphorous pentoxide, calcium carbonate, ferric oxide, or combinations thereof. An embodiment of the invention is one, any or all of prior embodiments in this paragraph up through the first embodiment in this paragraph wherein the first trapping agent comprises cerium, ceria or active alumina. An embodiment of the invention is one, any or all of prior embodiments in this paragraph up through the first embodiment in this paragraph wherein the first trapping agent is only in the core but not present in the outer shell. An embodiment of the invention is one, any or all of prior embodiments in this paragraph up through the first embodiment in this paragraph wherein the second trapping agent is only in the outer shell but not present in the core.

A second embodiment of the invention is a process for improving the yield of propylene from a light naphtha feedstock, comprising obtaining residual oil feedstock from an atmospheric or vacuum distillation tower, the residual oil feedstock comprising contaminant metals; contacting the residual oil feedstock with a cracking catalyst in a catalytic cracking zone to make a first slate of products, the cracking catalyst comprising a ZSM-5 zeolite, a core, an outer shell surrounding the core on the surface of the catalyst and a metal trap, wherein the metal trap comprises a first trapping agent in the core, a second trapping agent in an outer shell; separating the cracking catalyst from the first slate of products, including a light naphtha, in a first separator zone; contacting the light naphtha with the cracking catalyst to make a second set of products in an olefin-producing zone; separating the cracking catalyst from the second slate of products; regenerating the cracking catalyst by combusting coke deposited on a surface of the cracking catalyst in an oxygen-containing environment; and returning a first portion of the cracking catalyst to the catalytic cracking zone and a second portion of the cracking catalyst to the olefin-producing zone. An embodiment of the invention is one, any or all of prior embodiments in this paragraph up through the second embodiment in this paragraph further comprising a third trapping agent added to a bed of cracking catalyst. An embodiment of the invention is one, any or all of prior embodiments in this paragraph up through the second embodiment in this paragraph wherein the first trapping agent comprises cerium, ceria or active alumina. An embodiment of the invention is one, any or all of prior embodiments in this paragraph up through the second embodiment in this paragraph wherein the second trapping agent comprises calcium carbonate, ferric oxide, phosphorus pentoxide or combinations thereof. An embodiment of the invention is one, any or all of prior embodiments in this paragraph up through the second embodiment in this paragraph wherein the cracking catalyst further comprises a second zeolite component. An embodiment of the invention is one, any or all of prior embodiments in this paragraph up through the second embodiment in this paragraph wherein the first trapping agent is only in the core but not present in the outer shell. An embodiment of the invention is one, any or all of prior embodiments in this paragraph up through the second embodiment in this paragraph wherein the second trapping agent is only in the outer shell but not present in the core.

A third embodiment of the invention is a catalyst composition comprising a ZSM-5 zeolite, a core, an outer shell surrounding the core, and a metal trap, wherein the metal trap comprises a first trapping agent in the core and a second trapping agent in an outer shell of the catalyst. An embodiment of the invention is one, any or all of prior embodiments in this paragraph up through the third embodiment in this paragraph wherein the first trapping agent comprises cerium, ceria or active alumina. An embodiment of the invention is one, any or all of prior embodiments in this paragraph up through the third embodiment in this paragraph wherein the second trapping agent comprises calcium carbonate, ferric oxide, phosphorus pentoxide or combinations thereof. An embodiment of the invention is one, any or all of prior embodiments in this paragraph up through the third embodiment in this paragraph wherein the ZSM-5 zeolite is present only in the outer shell, but is not present in the core. An embodiment of the invention is one, any or all of prior embodiments in this paragraph up through the third embodiment in this paragraph wherein the first trapping agent is only in the core but not present in the outer shell. An embodiment of the invention is one, any or all of prior embodiments in this paragraph up through the third embodiment in this paragraph wherein the second trapping agent is only in the outer shell but not present in the core.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for improving the yield of propylene from a light naphtha feedstock, comprising:
obtaining residual oil feedstock from an atmospheric or vacuum distillation tower, the residual oil feedstock comprising contaminant metals;
contacting the residual oil feedstock with a cracking catalyst in a catalytic cracking zone to make a slate of products, the cracking catalyst comprising a ZSM-5 zeolite, a core, an outer shell comprising a layer surrounding the core on the surface of the catalyst and a metal trap, wherein the metal trap comprises a first trapping agent in the core, a second trapping agent in the outer shell;
separating the cracking catalyst from the products in a separator zone;
regenerating the cracking catalyst by combusting coke deposited on a surface of the cracking catalyst in an oxygen-containing environment; and
returning the cracking catalyst to the catalytic cracking zone.

2. The process of claim 1 further comprising a third trapping agent added to a bed of cracking catalyst.

3. The process of claim 2 wherein the shell further comprises an alumina wash coat.

4. The process of claim 3 wherein the second trapping agent comprises phosphorous pentoxide, calcium carbonate, ferric oxide, or combinations thereof.

5. The process of claim 1 wherein the first trapping agent comprises cerium, ceria or active alumina.

6. The process of claim 1 wherein the first trapping agent is only in the core but not present in the outer shell.

7. The process of claim 1 wherein the second trapping agent is only in the outer shell but not present in the core.

8. A process for improving the yield of propylene from a light naphtha feedstock, comprising:
obtaining residual oil feedstock from an atmospheric or vacuum distillation tower, the residual oil feedstock comprising contaminant metals;
contacting the residual oil feedstock with a cracking catalyst in a catalytic cracking zone to make a first slate of products, the cracking catalyst comprising a ZSM-5 zeolite, a core, an outer shell surrounding said core on the surface of the catalyst and a metal trap, wherein the metal trap comprises a first trapping agent in the core, a second trapping agent in an outer shell;
separating the cracking catalyst from the first slate of products, including a light naphtha, in a first separator zone;
contacting the light naphtha with the cracking catalyst to make a second set of products in an olefin-producing zone;
separating the cracking catalyst from the second slate of products;
regenerating the cracking catalyst by combusting coke deposited on a surface of the cracking catalyst in an oxygen-containing environment; and
returning a first portion of the cracking catalyst to the catalytic cracking zone and a second portion of the cracking catalyst to the olefin-producing zone.

9. The process of claim 8 further comprising a third trapping agent added to a bed of cracking catalyst.

10. The process of claim 8 wherein the first trapping agent comprises cerium, ceria or active alumina.

11. The process of claim 8 wherein the second trapping agent comprises calcium carbonate, ferric oxide, phosphorus pentoxide or combinations thereof

12. The process of claim 8 wherein the cracking catalyst further comprises a second zeolite component.

13. The process of claim 8 wherein the first trapping agent is only in the core but not present in the outer shell.

14. The process of claim 8 wherein the second trapping agent is only in the outer shell but not present in the core.

15. A catalyst composition comprising:
a ZSM-5 zeolite,
a core,
an outer shell surrounding the core, and
a metal trap, wherein the metal trap comprises a first trapping agent in the core and a second trapping agent in an outer shell of the catalyst.

16. The composition of claim 15 wherein the first trapping agent comprises cerium, ceria or active alumina.

17. The composition of claim 15 wherein the second trapping agent comprises calcium carbonate, ferric oxide, phosphorus pentoxide or combinations thereof

18. The composition of claim 15 wherein the ZSM-5 zeolite is present only in the outer shell, but is not present in the core.

19. The composition of claim 15 wherein the first trapping agent is only in the core but not present in the outer shell.

20. The composition of claim 15 wherein the second trapping agent is only in the outer shell but not present in the core.
